(19) United States
(12) Patent Application Publication
(10) Pub. No.: US 2025/0312253 A1
(43) Pub. Date: Oct. 9, 2025

(54) COMPOSITIONS AND METHODS FOR ALTERING THE COLOR OF HAIR

(71) Applicant: L'OREAL, Paris (FR)

(72) Inventors: Sarah Mary SPEER, Linden, NJ (US); Tathagata MUKHERJEE, Union, NJ (US); Shahid Mohammad NASEER, Hasbrouck Heights, NJ (US)

(21) Appl. No.: 18/626,300

(22) Filed: Apr. 3, 2024

(51) Int. Cl. A61K 8/46; A61K 8/06; A61K 8/365; A61K 8/41; A61Q 5/00

(52) U.S. Cl. CPC A61K 8/46; A61K 8/06; A61K 8/365; A61K 8/41; A61Q 5/00

(57) ABSTRACT

The disclosure relates to compositions and methods for altering the color of hair, the compositions comprising at least one color-removing agent, at least one surfactant, and at least one solvent. The compositions are in the form of emulsions.

### TECHNICAL FIELD

The disclosure relates to compositions and methods for altering the color, tone, and/or shade of hair. The compositions and methods can be used to alter the color of hair by removing various color, tones, and/or shades from the hair.

### BACKGROUND

Consumers often desire to change the color of their hair. For example, as a person ages, the hair follicle loses its natural pigment, resulting in grey, silver, or white hair. It is common for consumers to color their grey hairs, for example to match their natural hair color. Similarly, consumers may wish to change the color, tone, and/or shade of their hair from that of their natural color or a previous hair dye, e.g. may wish change to a different color entirely, may wish to lighten or darken their hair color, may wish to add warmth or coolness to the color of their hair, etc. The most common hair dyeing processes are permanent, semi-permanent, and temporary hair dyeing.

Semi-permanent or temporary hair dyeing compositions typically use pigments, liposoluble dyes, or direct dyes which are deposited onto the hair fiber to impart color to the hair. Generally, these hair dyes are washed out of the hair after one to several shampoo cycles.

On the other hand, permanent hair dyeing compositions uses oxidation dye precursors, which are also known as primary intermediates or couplers. In order to provide various colors, tones, and shades of permanent hair color, dye compositions contain various combinations of different types of oxidation dye compounds. These oxidation dye compounds are small, colorless or weakly colored compounds which, when combined with oxidizing agents, give rise to colored complexes by a process of oxidative condensation. The permanent hair dye compositions also contain ammonia or other alkalizing agents which causes the hair shaft to swell, thus allowing the oxidative dye molecules to penetrate the cuticle and cortex before the oxidation condensation process is completed. The resulting larger-sized colored complexes from the oxidative reaction are then trapped inside the hair fiber and cannot be washed out, thereby permanently altering the color of the hair.

Because permanent hair dyes are not able to be washed out of the hair, if an individual who has previously changed the color or tone of their hair with a first oxidative dye composition wishes to alter the color or tone of their previously-colored hair, some or all of the color imparted by the first oxidative dye composition must be neutralized. For example, a consumer may wish to remove the first color or tone in order to go back to their natural hair color, or to achieve a second color different from the first color by using a second oxidative dye composition. In such cases it is necessary to remove all of the first color from the hair, either to allow the natural hair color to be visible or to prevent the first color from interfering with the appearance of the second color. As another example, a consumer may wish to achieve a second tone or shade in the same color family as the first color, such as changing hair color from dark brown to light brown, and therefore may desire to remove some, but not necessarily all, of the first color. Alternatively, the change desired may be more subtle, for example by increasing the warmth or brightness of the first color. Thus, depending on the particular combination of oxidation dye compounds present in the first dye composition, and the desired second color or tone to be achieved, different levels and types of color removal are required.

Although there are known methods that are effective for removing the first color from the hair, these methods have drawbacks. For example, one common color-removal process uses oxidizing agents, but these compositions are harsh and lead to damage to the hair, causing the hair to be weak, brittle, and have an unhealthy appearance. This can be particularly problematic since the hair has already been subjected to harsh chemicals such as alkalizing agents and oxidizing agents in the first oxidative dye composition, and the integrity of the hair fibers is therefore already compromised. Moreover, these compositions can remove essentially all color from the hair, and therefore subsequent application of a second oxidative dye composition is needed to achieve the desired second color or tone. This step, known as “backfilling” the color, further exposes the hair to harsh chemicals and leads to additional damage.

In view of the damage associated with the use of oxidizing agents as color removers, a second option involves neutralizing the first oxidative dye molecules with color-removing agents such as thiol-based compounds. However, these compositions have not been as effective at removing color as those using oxidizing agents. For example, some oxidative dye compounds, particularly those imparting darker colors such as blacks, ash browns, browns, ash mochas, natural ash (blue or green), ash violets, violets, red violets, reds, red mochas, red browns, mahogany, red coppers, coppers, and golds, are more resistant to removal using thiol-based color-removing agents than to removal using oxidizing agents. As such, the color result achieved with these color-removing agents may not be satisfactory when used on hair that has been dyed with such compounds, and the remaining color will interfere with the colorist's attempt to achieve the desired color, tone, or shade.

Although attempts to solve these problems have been made, for example by adding components to the composition containing the oxidizing agent to minimize hair damage or by using different color-removing agents, to date there has not been a composition or process that is effective at removing oxidative color to permit a change in hair color, that adequately minimizes or eliminates damage to the hair. Thus, the choices that are available today for consumers who wish to alter the color of their previously-dyed hair are to either (1) remove all hair color with a color-removing composition containing damaging oxidizing agents, and subsequently expose the hair to further damage with a second oxidative dye composition to achieve the desired color or tone, or (2) remove only the hair dyes from the first color that are susceptible to removal with other color-removing agents such as thiol-based compounds, and attempt to achieve the desired color or tone with a second oxidative dye composition despite the remaining first color.

It has now been surprisingly discovered that the use of emulsions containing color-removing agents permits controlled removal of artificial color from previously-dyed hair, providing desired alterations in hair color, tone, and/or shade. The emulsion compositions are unique in that they can thus be used in color-removal processes to achieve a desired color, tone, and/or shade, much like consumers experience when undergoing a hair dye process, even in the absence of a backfilling step. Further, the emulsions are free or substantially free of oxidizing agents, and therefore minimize or eliminate damage to hair typically associated with hair color-removing compositions and processes.

### SUMMARY

The disclosure relates to emulsion compositions and methods that can be used to remove artificial color from the hair, in particular hair that has previously been dyed with an oxidative dye composition. In various embodiments, the compositions and methods can remove some or all of certain dyes, while having minimal or no impact on other dyes. As such, the color left on the hair can be “tuned” to achieve the consumer's desired result. The compositions and methods are able to achieve a desired hair color, tone, and/or shade even without, or with minimal, application of, subsequent hair coloring compositions. Surprisingly, the emulsion compositions have little to no odor, unlike currently-known hair-color removing compositions.

In various embodiments, the disclosure relates to emulsion compositions for treating keratin fibers such as hair, for example for removing color from the hair, the compositions comprising (a) at least one color-removing agent, (b) at least one surfactant, (c) at least one fatty compound, and (c) at least one solvent, where the total amounts of surfactants, fatty compounds, and water are sufficient to maintain the composition in the form of an emulsion. The compositions optionally comprise at least one additional compound chosen from clays, thickening agents, organic acids and/or salts thereof, amino acids and/or salts thereof, amino-sulfonic acids and/or salts thereof, organic amine compounds, or combinations of two or more thereof. The solvent may be chosen from water and/or non-aqueous solvents. The pH of the emulsion is acidic, for example ranging from about 1 to about 6, such as from about 2 to about 5.5, from about 2.5 to about 5, from about 2.75 to about 4.75, from about 3 to about 4.5, or from about 3 to about 4.

In various embodiments, the total amount of color-removing agents in the composition can range from about 0.1% to about 20%, such as from about 1% to about 18%, from 2% to about 15%, or from about 4% to about 12% by weight, relative to the total weight of the composition. In some embodiments, the color-removing agents are chosen from thiol-based color-removing agents, for example thiosalicylic acid, thiolactic acid, and/or salts thereof. In various embodiments, the total amount of thiosalicylic acid, thiolactic acid, and/or salts thereof, ranges from 1% to about 20%, from 2% to about 18%, or from about 4% to about 16% by weight, relative to the total weight of the composition.

Surfactants that can be chosen include nonionic, anionic, amphoteric, and cationic surfactants. In some embodiments, the compositions comprise at least one nonionic and/or at least one cationic surfactant. The total amount of surfactants will vary, but will be chosen to permit the composition to remain in the form of an emulsion. For example, the total amount of surfactants may range from about 0.5% to about 25%, such as from about 1% to about 20%, from about 1.5% to about 15%, or from about 2% to about 10% by weight, relative to the total weight of the composition.

Fatty compounds that can be used include, for example, lower alkanes, fatty alcohols, fatty acids, esters of fatty acids, esters of fatty alcohols, oils such as mineral, vegetable, animal, silicone and non-silicone oils, or combinations of any two or more thereof. The total amount of fatty compounds will vary, but will be chosen to permit the composition to remain in the form of an emulsion. For example, the total amount of fatty compounds may range from about 0.1% to about 15%, from about 0.25% to about 12%, from about 0.5% to about 10%, from about 0.75% to about 8%, or from about 1% to about 6% by weight, relative to the total weight of the composition.

In various embodiments, the emulsion compositions comprise one or more additional components chosen from organic amine compounds, organic acids and/or salts thereof, amino acids and/or salts thereof, amino sulfonic acids and/or salts thereof, thickening agents, clays, or combinations of two or more thereof. In some embodiments, the compositions comprise at least one keto acid and/or a salt thereof. In some embodiments, the compositions comprise at least one organic amine compound.

In further embodiments, the disclosure relates to methods of treating hair using the emulsion compositions according to the disclosure. For example, the methods may be methods of removing dyes, e.g. oxidative dyes, from the hair. In some embodiments, a composition according to the disclosure may be applied to the hair and left on the hair for a period of time, for example ranging from about 1 minute to about 1 hour, from about 1 minute to about 45 minutes, from about 1 minute to about 30 minutes, from about 5 minutes to about 1 hour, from about 5 minutes to about 45 minutes, from about 5 minutes to about 30 minutes, etc. The hair may optionally be heated during the leave-in period. The methods may further comprise removing the compositions from the hair, e.g. by rinsing and/or shampooing the hair.

### DETAILED DESCRIPTION

The disclosure relates to emulsion compositions and methods for altering the color of the hair, in particular hair that has been previously dyed with an oxidative dye composition. The methods comprise removing some or all of various dye compounds in order to provide a desired color, tone, and/or shade of the treated hair.

### I. Compositions

Compositions according to the disclosure are in the form of an emulsion and comprise (a) at least one color-removing agent, (b) at least one surfactant, (c) at least one fatty compound, and (d) at least one solvent, and optionally at least one additional compound chosen from clays, thickeners, organic acids, amino acids, organic amine compounds, or combinations of two or more thereof.

### Color-Removing Agents

Emulsion compositions according to the disclosure comprise at least one color-removing agent. Color-removing agents according to the disclosure are not oxidizing agents. By way of example, useful color-removing agents may be chosen from cysteine, homocysteine, thiolactic acid, thioglycolic acid, glutaric acid, glyoxylic acid, glycolic acid, citric acid, ascorbic acid, salts thereof, derivatives thereof, or combinations of two or more thereof. Non-limiting examples of salts include sodium salts, ammonium salts, lithium salts, potassium salts, and calcium salts.

In some embodiments, the color-removing agents chosen comprise, consist essentially of, or consist of thiol-based compounds. Exemplary thiol-based color-removing agents include thiolactic acid and/or salts thereof, thioglycolic acid and/or esters thereof, thiosalicylic acid and/or salts thereof, or combinations of two or more thereof.

Emulsion compositions according to the disclosure may optionally comprise one or more additional color-removing agents. For example, the emulsion compositions may optionally include at least one additional color-removing agent chosen from borohydrides and/or derivatives thereof, phosphines and/or salts thereof, bisulphites and/or salts thereof, sulphites and/or salts thereof, or combinations of two or more thereof. However, in some embodiments if such additional color-removing agent(s) are present, the total amount of additional color-removing agents may be less than about 5%, less than about 4.5%, less than about 4%, less than about 3.5%, less than about 3%, less than about 2.5%, less than about 2%, less than about 1.5%, less than about 1%, less than about 0.5%, or less than about 0.1% by weight of the composition.

In various embodiments, the total amount of color-removing agents may range from about 0.5% to about 25%, such as from about 1% to about 22%, from 2% to about 20%, or from about 3% to about 18% by weight, relative to the total weight of the composition. For example, in various embodiments, the total amount of color-removing agents may range from about 1% to about 25%, from about 1% to about 22%, from about 1% to about 20%, from about 1% to about 18%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, about 2% to about 25%, from about 2% to about 22%, from about 2% to about 20%, from about 2% to about 18%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, about 3% to about 25%, from about 3% to about 22%, from about 3% to about 20%, from about 3% to about 18%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, about 4% to about 25%, from about 4% to about 22%, from about 4% to about 20%, from about 4% to about 18%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, about 5% to about 25%, from about 5% to about 22%, from about 5% to about 20%, from about 5% to about 18%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6%, about 6% to about 25%, from about 6% to about 22%, from about 6% to about 20%, from about 6% to about 18%, from about 6% to about 15%, from about 6% to about 14%, from about 6% to about 13%, from about 6% to about 12%, from about 6% to about 11%, from about 6% to about 10%, from about 6% to about 9%, from about 6% to about 8%, from about 6% to about 7%, about 7% to about 25%, from about 7% to about 22%, from about 7% to about 20%, from about 7% to about 18%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, about 8% to about 25%, from about 8% to about 22%, from about 8% to about 20%, from about 8% to about 18%, from about 8% to about 15%, from about 8% to about 14%, from about 8% to about 13%, from about 8% to about 12%, from about 8% to about 11%, from about 8% to about 10%, from about 8% to about 9%, about 9% to about 25%, from about 9% to about 22%, from about 9% to about 20%, from about 9% to about 18%, from about 9% to about 15%, from about 9% to about 14%, from about 9% to about 13%, from about 9% to about 12%, from about 9% to about 11%, from about 9% to about 10%, about 10% to about 25%, from about 10% to about 22%, from about 10% to about 20%, from about 10% to about 18%, from about 10% to about 15%, from about 10% to about 14%, from about 10% to about 13%, from about 10% to about 12%, or from about 10% to about 11% by weight, relative to the total weight of the composition. For example, the total amount of color-removing agents may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, abut 19%, or about 20% by weight, relative to the total weight of the composition, including any range using any of the foregoing as upper and lower limits.

In some embodiments, the compositions comprise a total amount of thiol-based color-removing agents in any of the foregoing ranges or amounts. For example, the compositions may comprise one or more of thiosalicylic acid, thiolactic acid, and/or salts of any of the foregoing as color-removing agents, and the total amount of thiosalicylic acid, thiolactic acid, and salts of any of the foregoing may be at least about 1%, for example may range from about 1% to about 25%, from about 1% to about 22%, from about 1% to about 20%, from about 1% to about 18%, from about 1% to about 15%, from about 1% to about 12%, from about 1% to about 10%, from about 1% to about 8%, from about 1% to about 6%, from about 1.5% to about 25%, from about 1.5% to about 22%, from about 1.5% to about 20%, from about 1.5% to about 18%, from about 1.5% to about 15%, from about 1.5% to about 12%, from about 1.5% to about 10%, from about 1.5% to about 8%, from about 1.5% to about 6%, from about 2% to about 25%, from about 2% to about 22%, from about 2% to about 20%, from about 2% to about 18%, from about 2% to about 15%, from about 2% to about 12%, from about 2% to about 10%, from about 2% to about 8%, from about 2% to about 6%, from about 2.5% to about 20%, from about 2.5% to about 18%, from about 2.5% to about 15%, from about 2.5% to about 12%, from about 2.5% to about 10%, from about 2.5% to about 8%, from about 2.5% to about 6%, from about 3% to about 25%, from about 3% to about 22%, from about 3% to about 20%, from about 3% to about 18%, from about 3% to about 15%, from about 3% to about 12%, from about 3% to about 10%, from about 3% to about 8%, from about 3% to about 6%, from about 3.5% to about 25%, from about 3.5% to about 22%, from about 3.5% to about 20%, from about 3.5% to about 18%, from about 3.5% to about 15%, from about 3.5% to about 12%, from about 3.5% to about 10%, from about 3.5% to about 8%, from about 3.5% to about 6%, from about 4% to about 25%, from about 4% to about 22%, from about 4% to about 20%, from about 4% to about 18%, from about 4% to about 15%, from about 4% to about 12%, from about 4% to about 10%, from about 4% to about 8%, from about 4% to about 6%, from about 4.5% to about 25%, from about 4.5% to about 22%, from about 4.5% to about 20%, from about 4.5% to about 18%, from about 4.5% to about 15%, from about 4.5% to about 12%, from about 4.5% to about 10%, from about 4.5% to about 8%, or from about 4.5% to about 6% by weight, relative to the total weight of the composition. For example, the composition may comprise a total amount of thiol-based color-removing agents, e.g. a total among of thiosalicylic acid, thiolactic acid, and salts thereof, of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% by weight, relative to the total weight of the composition, or may be present in a range using any of the foregoing as upper or lower limits. In such embodiments, the compositions may optionally comprise at least one additional color-removing agent in addition to thiol-based agents.

### Surfactants

Emulsion compositions according to the disclosure comprise at least one surfactant, which may in various embodiments be chosen from anionic, non-ionic, amphoteric, and/or cationic surfactants. For example, the emulsion compositions may comprise one or more anionic surfactants, one or more non-ionic surfactants, or one or more amphoteric surfactants, one or more cationic surfactants, or the emulsion compositions may comprise mixtures of surfactants having the same or different ionicities. Salts of surfactants are included whether or not expressly stated, unless specified otherwise.

The total amount of surfactants may vary, but will be sufficient, in combination with other components, to provide an emulsion composition. For example, the total amount of surfactants may range from about 0.5% to about 25%, such as from about 1% to about 20%, from about 1.5% to about 15%, or from about 2% to about 10% by weight, relative to the total weight of the composition. In some exemplary embodiments, the total amount of surfactants may range from about 0.5% to about 12%, from about 0.5% to about 10%, from about 0.5% to about 8%, from about 0.5% to about 6%, from about 1% to about 12%, from about 1% to about 10%, from about 1% to about 8%, from about 1% to about 6%, from about 1.5% to about 12%, from about 1.5% to about 10%, from about 1.5% to about 8%, from about 1.5% to about 6%, from about 2% to about 12%, from about 2% to about 10%, from about 2% to about 8%, from about 2% to about 6% by weight, relative to the total weight of the composition. For example, the total amount of surfactants may be about 0.5%, about 1%, about 1.5%, about 2%, about 2.5%, about 3%, about 3.5%, about 4%, about 4.5%, about 5%, about 5.5%, about 6%, about 6.5%, or about 7% by weight, relative to the total weight of the composition, or may be present in any range using any of the foregoing as upper and lower limits.

### Anionic Surfactants

In at least some embodiments, the emulsion compositions comprise at least at least one anionic surfactant. “Anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from, for example, —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate, and/or carboxylic (or carboxylate) surfactants, or mixtures thereof.

Sulfate anionic surfactants comprise at least one sulfate function (—OSO₃H or —OSO₃) but generally do not comprise any carboxylate or sulfonate functions. In some embodiments the sulfate anionic surfactants that are used are free of carboxylate or sulfonate functions.

Non-limiting examples of sulfate anionic surfactants include alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, for example polyoxyethylenated, and optionally comprising from 1 to 50 ethylene oxide units such as from 2 to 10 ethylene oxide units.

Sulfonate anionic surfactants comprise at least one sulfonate function (—SO₃H or —SO₃⁻) and may optionally also comprise one or more sulfate functions, but preferably do not comprise any carboxylate functions.

Non-limiting examples of sulfonate anionic surfactants include alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

Carboxylate anionic surfactants comprise at least one carboxylic or carboxylate function (—COOH or —COO⁻) and may optionally also comprise one or more sulfate and/or sulfonate functions.

Non-limiting examples of carboxylate anionic surfactants include acylglycinates, acyllactylates, acylsarcosinates, acylglutamates, alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

When the anionic surfactant is in salt form, the salt may be chosen from alkali metal salts, such as the sodium or potassium salt, ammonium salts, amine salts and in particular amino alcohol salts, and alkaline-earth metal salts, such as the magnesium salt.

Examples of amino alcohol salts may include but are not limited to monoethanolamine, diethanolamine, and triethanolamine salts, monoisopropanolamine, diisopropanolamine, and triisopropanolamine salts, 2-amino-2-methyl-1-propanol salts, 2-amino-2-methyl-1,3-propanediol salts, and tris(hydroxymethyl)aminomethane salts.

In some embodiments, alkali metal or alkaline-earth metal salts and in particular the sodium or magnesium salts may be chosen, for example in the form of alkali metal, ammonium, amino alcohol and alkaline-earth metal salts, or a mixture of these compounds, may be chosen.

In exemplary and non-limiting embodiments, the anionic surfactant may be chosen from sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C₁₄₋₁₆ olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, sodium cocoyl glutamate, disodium cocoyl glutamate, potassium myristoyl glutamate, TEA-cocoyl glutamate, sodium cocoyl glycinate, potassium cocoyl glycinate, sodium cocoyl alaninate, TEA-cocoyl alaninate, or a combination of two or more thereof. For example, the emulsion compositions may comprise at least one anionic surfactant chosen from sodium laureth sulfate, sodium lauryl sulfate, sodium lauroyl sulfate, sodium lauroyl methyl isethionate, or a combination of two or more thereof.

If present, the total amount of anionic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of anionic surfactants may range from about 0.01% to about 12%, from about 0.1% to about 10%, from about 0.5% to about 8%, or from about 1% to about 6% by weight, relative to the total weight of the composition. In at least some embodiments, the emulsion compositions comprise at least one anionic surfactant, and have a total amount of anionic surfactants ranging from about 0.5% to about 10%, from about 0.5% to about 8%, from about 0.5% to about 6%, from about 0.5% to about 5%, from about 0.5% to about 4%, from about 0.5% to about 3%, from about 0.75% to about 10%, from about 0.75% to about 8%, from about 0.75% to about 6%, from about 0.75% to about 5%, from about 0.75% to about 4%, from about 0.75% to about 3%, from about 1% to about 10%, from about 1% to about 8%, from about 1% to about 6%, from about 1% to about 5%, from about 1% to about 4%, from about 1% to about 3%, from about 1.25% to about 10%, from about 1.25% to about 8%, from about 1.25% to about 6%, from about 1.25% to about 5%, from about 1.25% to about 4%, from about 1.25% to about 3%, from about 1.5% to about 10%, from about 1.5% to about 8%, from about 1.5% to about 6%, from about 1.5% to about 5%, from about 1.5% to about 4%, or from about 1.5% to about 3%, by weight relative to the total weight of the composition. In various embodiments, the total amount of anionic surfactant may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, or may be present in any range using any of the foregoing as upper and lower limits.

### Amphoteric Surfactants

In at least some embodiments, the emulsion compositions comprise at least one amphoteric (also referred to as zwitterionic surfactants), although in at least some embodiments the emulsion compositions are free or essentially free of amphoteric surfactants. Non-limiting examples of useful amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanolamine cocoamphohydroxypropylsulfonate, triethanonlamine cocoamphopropionate, triethanonlamine cornamphopropionate, triethanonlamine lauraminopropionate, triethanolamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as combinations of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, cocamidopropyl betaine, or combinations of two or more thereof, may be chosen.

If present, the total amount of amphoteric surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of amphoteric surfactants may range from about 0.01% to about 10%, from about 0.1% to about 8%, from about 0.5% to about 6%, or from about 1% to about 4% by weight, relative to the total weight of the composition. In at least some embodiments, the emulsion compositions comprise at least one amphoteric surfactant, and have a total amount of amphoteric surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In at least some other embodiments, the compositions are free or substantially free of amphoteric surfactants.

### Nonionic Surfactants

In at least some embodiments, the emulsion compositions comprise at least one nonionic surfactant, although in at least some embodiments the emulsion compositions are free or essentially free of nonionic surfactants. The nonionic surfactants may be chosen from alcohols, α-diols and (C₁-C₂₀) alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30, or alternatively these compounds comprising at least one fatty chain comprising from 8 to 30 carbon atoms and especially from 16 to 30 carbon atoms. For example, nonionic surfactants may be chosen from monooxyalkylenated or polyoxyalkylenated (C₈-C₂₄) alkylphenols, saturated or unsaturated, linear or branched, monooxyalkylenated or polyoxyalkylenated C₈-C₃₀ alcohols, saturated or unsaturated, linear or branched, monooxyalkylenated or polyoxyalkylenated C₈-C₃₀ amides, esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol, saturated or unsaturated, monooxyalkylenated or polyoxyalkylenated plant oils, condensates of ethylene oxide and/or of propylene oxide, or combinations thereof.

By way of example only, the adducts of ethylene oxide with lauryl alcohol, for example those containing from 9 to 50 oxyethylene units or from 10 to 12 oxyethylene units (Laureth-10 to Laureth-12); the adducts of ethylene oxide with behenyl alcohol, for example those containing from 9 to 50 oxyethylene units (Beheneth-9 to Beheneth-50); the adducts of ethylene oxide with cetearyl alcohol (mixture of cetyl alcohol and stearyl alcohol), for example those containing from 10 to 30 oxyethylene units (Ceteareth-10 to Ceteareth-30); the adducts of ethylene oxide with cetyl alcohol, for example those containing from 10 to 30 oxyethylene units (Ceteth-10 to Ceteth-30); the adducts of ethylene oxide with stearyl alcohol, for example those containing from 10 to 30 oxyethylene units (Steareth-10 to Steareth-30); the adducts of ethylene oxide with isostearyl alcohol, for example those containing from 10 to 50 oxyethylene units (Isosteareth-10 to Isosteareth-50); monoglycerolated or polyglycerolated C₈-C₄₀, e.g. C₈-C₃₀, alcohols, such as lauryl alcohol containing 4 mol of glycerol (Polyglyceryl-4 Lauryl Ether), lauryl alcohol containing 1.5 mol of glycerol, oleyl alcohol containing 4 mol of glycerol (Polyglyceryl-4 Oleyl Ether), oleyl alcohol containing 2 mol of glycerol (Polyglyceryl-2 Oleyl Ether), cetearyl alcohol containing 2 mol of glycerol, cetearyl alcohol containing 6 mol of glycerol, oleocetyl alcohol containing 6 mol of glycerol, or octadecanol containing 6 mol of glycerol; polyoxyethylenated fatty esters such as the adducts of ethylene oxide with esters of lauric acid, palmitic acid, stearic acid or behenic acid, and mixtures thereof, for example those containing from 9 to 100 oxyethylene units such as PEG-9 to PEG-50 laurate, PEG-9 to PEG-50 palmitate, PEG-9 to PEG-50 stearate, PEG-9 to PEG-50 palmitostearate, PEG-9 to PEG-50 behenate, polyethylene glycol 100 EO monostearate (PEG-100 stearate); glyceryl stearate (glyceryl mono-, di- and/or tristearate); glyceryl ricinoleate; sorbitan palmitate; sorbitan isostearate; sorbitan stearate; sorbitan palmitate; sorbitan trioleate; alkylglucose sesquistearates such as methylglucose sesquistearatel alkylglucose palmitates such as methylglucose or ethylglucose palmitate, etc., or combinations of two or more thereof may be chosen.

If present, the total amount of nonionic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of nonionic surfactants may range from about 0.01% to about 10%, from about 0.1% to about 8%, from about 0.5% to about 6%, or from about 1% to about 4% by weight, relative to the total weight of the composition. In at least some embodiments, the emulsion compositions comprise at least one nonionic surfactant, and have a total amount of nonionic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In at least some embodiments, the compositions are free or substantially free of nonionic surfactants.

### Cationic Surfactants

In at least some embodiments, the emulsion compositions comprise at least one cationic surfactant, although in at least some embodiments the emulsion compositions are free or essentially free of cationic surfactants. Exemplary and non-limiting cationic surfactants include cationic amine-based or quaternary ammonium-based compounds.

For example, cationic surfactants may be chosen from alkylpyridinium salts, ammonium salts of imidazoline, diquaternary ammonium salts, and ammonium salts containing at least one ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

Additional non-limiting examples of useful cationic surfactants include brassicamidopropyl dimethylamine, behentrimonium chloride, cetrimonium chloride, behenalkonium chloride, chloride, benzethonium cetylpyridinium chloride, behentrimonium chloride, lauralkonium chloride, cetalkonium chloride, cetrimonium bromide, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, stearamidopropyl dimethylamine, or combinations thereof.

If present, the total amount of cationic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of cationic surfactants may range from about 0.01% to about 10%, from about 0.1% to about 8%, from about 0.5% to about 6%, or from about 1% to about 4% by weight, relative to the total weight of the composition. In at least some embodiments, the emulsion compositions comprise at least one cationic surfactant, and have a total amount of cationic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In some embodiments, however, the compositions are free or essentially free of cationic surfactants.

### Fatty Compounds

Emulsion compositions according to the disclosure include at least one fatty compound. In various embodiments, the at least one fatty compound may be chosen from lower alkanes, fatty alcohols, fatty acids, esters of fatty acids, esters of fatty alcohols, oils such as mineral, vegetable, animal, silicone and non-silicone oils, silicone and non-silicone waxes, or combinations of any two or more thereof. In some embodiments, the emulsion compositions comprise at least one fatty compound of natural origin.

If present, silicone oils may, for example, be chosen from polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyl-diphenyl)trisiloxanes, or (2-phenylethyl)trimethyl-siloxysilicates. Non-limiting examples of silicone oils include dimethicone, amodimethicone, cyclomethicone, polysilicone-11, phenyl trimethicone, trimethylsilylamodimethicone, and stearoxytrimethylsilane. For example, the emulsion composition may comprise at least one silicone chosen from amodimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, Dimethicone PEG-8 Benzoate, Dimethicone PEG-7 Phosphate, Dimethicone PEG-8 Phosphate, Dimethicone PEG-10 Phosphate, or a combination of two or more thereof.

In some embodiments, the emulsion compositions comprise a silicone oil component that comprises, consists essentially of, or consists of dimethicone, amodimethicone, or a combination thereof. In some embodiments, the compositions are free or substantially free of silicone oils and/or silicone waxes.

In some embodiments, emulsion compositions according to the disclosure may include at least one fatty compound chosen from fatty alcohols. Herein, “fatty alcohol” can refer to any alcohol with a carbon chain of C5 or greater, such as, for example, C8 or greater, C10 or greater, or C12 or greater, such as from 6 to 30 carbon atoms or from 8 to 30 carbon atoms. The fatty alcohols may be alkoxylated or non-alkoxylated, saturated or unsaturated, and linear or branched. Non-limiting examples of fatty alcohols include arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof. In some embodiments, the compositions comprise a fatty alcohol component that comprises, consists essentially of, or consists of cetyl alcohol, stearyl alcohol, cetearyl alcohol, or combinations thereof.

Useful and non-limiting fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, or combinations thereof.

In some embodiments, fatty acid esters or fatty alcohol esters may be chosen. For example, esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10 may be used. In other embodiments, esters of C4-C22 dicarboxylic or tricarboxylic acids and of C1-C22 alcohols and esters of mono-, di- or tricarboxylic acids and of C2-C26 di-, tri-, tetra-, or pentahydroxy alcohols may also be used. As non-limiting examples, isostearyl lactate, lauryl lactate, linoleyl lactate, oleyl lactate, (iso) stearyl octanoate, isocetyl octanoate, octyl octanoate, cetyl octanoate, decyl oleate, isocetyl isostearate, isocetyl laurate, isocetyl stearate, isodecyl octanoate, isodecyl oleate, isononyl isononanoate, isostearyl palmitate, methyl acetyl ricinoleate, myristyl stearate, octyl isononanoate, 2-ethylhexyl isononanoate, octyl palmitate, octyl pelargonate, octyl stearate, octyldodecyl erucate, oleyl erucate, ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate, dioctyl malate, hexyl laurate, 2-hexyldecyl laurate, diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and/or polyethylene glycol distearates may be chosen. In an embodiment, the emulsion composition comprises at least one fatty acid ester and/or fatty alcohol ester.

Non-limiting examples of waxes that can be used include beeswax, hydrogenated alkyl olive esters, carnauba wax, candelilla wax, ouricoury wax, Japan wax, cork fibre wax or sugar cane wax, rice wax, rice bran wax, montan wax, paraffin wax, lignite wax or microcrystalline wax, ceresin or ozokerite, palm kernel glycerides/hydrogenated palm glycerides, palm butter, sumac wax, Citrus aurantium dulcis (orange) peel wax, Theobroma grandiflorum seed butter, Helianthus annuus (sunflower) seed wax, siliconyl candellila wax, Chinese wax, cetyl palmitate, lanolin, shellac, spermaceti, cetyl esters, hydrogenated castor wax; triglyceride esters such as tribehenin (glyceryl tribehenate); synthetic waxes such as those of the hydrocarbon type and polyethylene waxes obtained from the polymerization or copolymerization of ethylene, polypropylene waxes, or combinations of two or more of any of these waxes. However, in some embodiments the compositions are free or essentially free of waxes.

In some embodiments, the emulsion composition may include one or more fatty compounds chosen from oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, fluorosilicone oils, or combinations of two or more thereof. In some embodiments, the emulsion compositions comprise at least one oil, e.g. mineral oil.

The total amount of fatty compounds will be chosen to permit the composition to remain in the form of an emulsion. For example, the total amount of fatty compounds in the emulsion composition may range from about 0.01% up to about 20%, such as up to about 18%, up to about 15%, or up to about 12% by weight, relative to the total weight of the composition. In some exemplary embodiments, the total amount of fatty compounds may range from about 0.1% to about 20%, from about 0.1% to about 18%, from about 0.1% to about 15%, from about 0.1% to about 12%, from about 0.1% to about 10%, from about 0.1% to about 8%, from about 0.1% to about 6%, from about 0.5% to about 20%, from about 0.5% to about 18%, from about 0.5% to about 15%, from about 0.5% to about 12%, from about 0.5% to about 10%, from about 0.5% to about 8%, from about 0.5% to about 6%, from about 1% to about 20%, from about 1% to about 18%, from about 1% to about 15%, from about 1% to about 12%, from about 1% to about 10%, from about 1% to about 8%, from about 1% to about 6%, from about 2% to about 20%, from about 2% to about 18%, from about 2% to about 15%, from about 2% to about 12%, from about 2% to about 10%, from about 2% to about 8%, or from about 2% to about 6% by weight, relative to the total weight of the composition. For example, the total amount of fatty compounds may be about 1%, about 2%, about 3%, about 4%, about 5%, about 6%, about 7%, about 8%, about 9%, about 10%, about 11%, about 12%, about 13%, about 14%, or about 15% by weight, relative to the total weight of the composition, including any range using any of the foregoing as upper and lower limits.

### Solvents

Emulsion compositions according to the disclosure comprise at least one solvent. The solvent may be chosen from water, non-aqueous solvents, or combinations thereof. However, the solvent will be chosen to permit the composition to remain in the form of an emulsion.

In some embodiments, the solvent comprises, consists essentially of, or consists of water. The total amount of water may vary depending on the desired properties of composition, for example consistency, viscosity, etc.

In some embodiments, non-aqueous solvents may be used, for example, glycerin, C₁₋₄ alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

The total amount of solvents in the composition typically ranges from about 50% to about 98%, such as from about 60% to about 98%, from about 70% to about 98%, from about 75% to about 98%, from about 75% to about 95%, or from about 80% to about 90% by weight, relative to the total weight of the composition.

### Organic Acids

Emulsion compositions according to the disclosure optionally comprise at least one organic acid, for example chosen from carboxylic acids, which includes, for example, α-hydroxy acids and/or keto acids. Salts of organic acids may also be chosen and are expressly included unless stated otherwise.

In various embodiments, useful organic acids include citric acid, maleic acid, succinic acid, aspartic acid, glutamic acid, lactic acid, malic acid, tartaric acid, glyceric acid, gluconic acid, glutaric acid, acetic acid, glycolic acid, oxalic acid, 2-ketobutyric acid, β-hydroxypyruvic acid, ketomalonic acid, oxoacetic acid, 2-ketoglutaric acid, 2-keto-L-gulonic acid, 2-ketoglutaric acid dihydrate, pyruvamide salts thereof, or combinations thereof. In some embodiments, the emulsion composition comprises one or more organic acids chosen from 2-oxoglutaric acid (ketoglutaric acid), glyoxylic acid, glycolic acid, maleic acid, citric acid, lactic acid, acetic acid, salts thereof, or combinations of two or more thereof.

In some embodiments, the emulsion compositions comprise at least one carboxylic acid. In some embodiments, the emulsion compositions comprise at least one keto acid. Without intending to be bound by theory, it is believed that keto acids, which are acids containing two oxygen-containing functional groups, a carboxylic acid (—COOH) and a ketone group (>C═O), increase color-removal benefits of color-removing agents.

If present, the total amount of organic acids and salts thereof may range from about 0.01% up to about 40%, such as up to about 38%, up to about 35%, up to about 30%, up to about 28%, up to about 25%, up to about 20%, up to about 15%, up to about 13%, up to about 10%, up to about 9%, up to about 8%, up to about 7%, up to about 6%, or up to about 5% by weight, relative to the total weight of the composition.

For example, the emulsion compositions may comprise at least one carboxylic acid and the total amount of carboxylic acids may range from about 0.1% to about 30%, from about 0.1% to about 28%, from about 0.1% to about 25%, from about 0.1% to about 20%, from about 0.1% to about 15%, from about 0.1% to about 13%, from about 0.1% to about 10%, from about 0.1% to about 8%, from about 0.1% to about 6%, from about 0.1% to about 5%, from about 0.5% to about 30%, from about 0.5% to about 28%, from about 0.5% to about 25%, from about 0.5% to about 20%, from about 0.5% to about 15%, from about 0.5% to about 13%, from about 0.5% to about 10%, from about 0.5% to about 8%, from about 0.5% to about 6%, from about 0.5% to about 5%, from about 1% to about 30%, from about 1% to about 28%, from about 1% to about 25%, from about 1% to about 20%, from about 1% to about 15%, from about 1% to about 13%, from about 1% to about 10%, from about 1% to about 8%, from about 1% to about 6%, from about 1% to about 5%, from about 1.5% to about 30%, from about 1.5% to about 28%, from about 1.5% to about 25%, from about 1.5% to about 20%, from about 1.5% to about 15%, from about 1.5% to about 13%, from about 1.5% to about 10%, from about 1.5% to about 8%, from about 1.5% to about 6%, from about 1.5% to about 5%, from about 2% to about 30%, from about 2% to about 28%, from about 2% to about 25%, from about 2% to about 20%, from about 2% to about 15%, from about 2% to about 13%, from about 2% to about 10%, from about 2% to about 8%, from about 2% to about 6%, from about 2% to about 5%, from about 3% to about 30%, from about 3% to about 28%, from about 3% to about 25%, from about 3% to about 20%, from about 3% to about 15%, from about 3% to about 13%, from about 3% to about 10%, from about 3% to about 8%, from about 3% to about 6%, from about 3% to about 5%, from about 4% to about 30%, from about 4% to about 28%, from about 4% to about 25%, from about 4% to about 20%, from about 4% to about 15%, from about 4% to about 13%, from about 4% to about 10%, from about 4% to about 8%, from about 5% to about 30%, from about 5% to about 28%, from about 5% to about 25%, from about 5% to about 20%, from about 5% to about 15%, from about 5% to about 13%, from about 5% to about 10%, from about 5% to about 8%, from about 6% to about 30%, from about 6% to about 28%, from about 6% to about 25%, from about 6% to about 20%, from about 6% to about 15%, from about 6% to about 13%, from about 6% to about 10%, from about 6% to about 8%, from about 8% to about 30%, from about 8% to about 28%, from about 8% to about 25%, from about 8% to about 20%, from about 8% to about 15%, from about 8% to about 13%, from about 10% to about 30%, from about 10% to about 28%, from about 10% to about 25%, from about 10% to about 20%, from about 10% to about 15%, from about 10% to about 13%, from about 12% to about 30%, from about 12% to about 28%, from about 12% to about 25%, from about 12% to about 20%, from about 12% to about 15%, or from about 12% to about 13% by weight, relative to the total weight of the composition. In some embodiments, the total amount of carboxylic acids may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, or about 25% by weight, relative to the total weight of the composition, including all ranges and subranges using any of the foregoing as upper and lower limits.

In other embodiments, the emulsion compositions comprise at least one keto acid, and the total amount of keto acids ranges from about 0.01% up to about 20%, up to about 15%, up to about 13%, up to about 10%, up to about 9%, up to about 8%, up to about 7%, up to about 6%, or up to about 5% by weight, relative to the total weight of the composition. For example, the total amount of keto acids may range from about 0.1% to about 20%, from about 0.1% to about 15%, from about 0.1% to about 13%, from about 0.1% to about 10%, from about 0.1% to about 8%, from about 0.5% to about 20%, from about 0.5% to about 15%, from about 0.5% to about 13%, from about 0.5% to about 10%, from about 0.5% to about 8%, from about 1% to about 20%, from about 1% to about 15%, from about 1% to about 13%, from about 1% to about 10%, from about 1% to about 8%, from about 1.5% to about 20%, from about 1.5% to about 15%, from about 1.5% to about 13%, from about 1.5% to about 10%, from about 1.5% to about 8%, from about 2% to about 20%, from about 2% to about 15%, from about 2% to about 13%, from about 2% to about 10%, from about 2% to about 8%, from about 3% to about 20%, from about 3% to about 15%, from about 3% to about 13%, from about 3% to about 10%, from about 3% to about 8%, from about 4% to about 20%, from about 4% to about 15%, from about 4% to about 13%, from about 4% to about 10%, from about 4% to about 8%, from about 5% to about 20%, from about 5% to about 15%, from about 5% to about 13%, from about 5% to about 10%, from about 5% to about 8%, from about 6% to about 20%, from about 6% to about 15%, from about 6% to about 13%, from about 6% to about 10%, from about 6% to about 8%, from about 8% to about 20%, from about 8% to about 15%, from about 8% to about 13%, from about 8% to about 12%, from about 8% to about 11%, from about 9% to about 20%, from about 9% to about 15%, from about 9% to about 13%, from about 9% to about 12%, or from about 9% to about 11% by weight, relative to the total weight of the composition. In some embodiments, the total amount of keto acids may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the composition, including all ranges and subranges using any of the foregoing as upper and lower limits. A preferred keto acid is ketoglutaric acid.

It may, in some embodiments, be advantageous to include at least one keto acid in the emulsion composition in an amount relative to the color-removing agent to maximize the color-removal benefits. Thus, in various embodiments, the emulsion compositions comprise at least one keto acid where the weight ratio of total amounts of color-removing agent(s):keto acid(s) ranges from about 1:0.5 to 1:10. In other embodiments, it may be advantageous for the weight ratio of total amounts of color-removing agent(s):keto acid(s) to be equal to or less than 1. For example, the weight ratio of total amounts of color-removing agent(s):keto acid(s) may range from about 1:1 to about 1:10, such as from about 1:1 to about 1:8, from about 1:1 to about 1:6, from about 1:1 to about 1:5, from about 1:1 to about 1:4, from about 1:1 to about 1:3, from about 1:1 to about 1:2, from about 1:1 to about 1:1.5, from about 1:1.1 to about 1:10, about 1:1.1 to about 1:8, from about 1:1.1 to about 1:6, from about 1:1.1 to about 1:5, from about 1:1.1 to about 1:4, from about 1:1.1 to about 1:3, from about 1:1.1 to about 1:2, or from about 1:1.1 to about 1:1.5. In other embodiments the weight ratio of total amounts of color-removing agent(s):keto acid(s) may be about 0.1, about 0.2, about 0.3, about 0.4, about 0.5, about 0.6, about 0.7, about 0.8, about 0.9, about 1, about 1.1, about 1.2, about 1.3, about 1.4, or about 1.5, including all ranges and subranges using any of the foregoing as upper and lower limits.

In embodiments where the color-removing agents are chosen from thiol-based compounds, e.g. thiosalicylic acid, thiolactic acid, and/or salts thereof, the weight ratio of total amounts of thiol-based color-removing agent(s):keto acid(s) can be any of the aforementioned ratios. For example, in some embodiments the weight ratio of thiol-based color-removing agent(s):keto acid(s) can range from about 1:0.5 to 1:10, or can be equal to or less than 1. For example, the weight ratio of total amounts of thiol-based color-removing agent(s):keto acid(s) may range from about 1:1 to about 1:10, such as from about 1:1 to about 1:8, from about 1:1 to about 1:6, from about 1:1 to about 1:5, from about 1:1 to about 1:4, from about 1:1 to about 1:3, from about 1:1 to about 1:2, from about 1:1 to about 1:1.5, from about 1:1.1 to about 1:10, about 1:1.1 to about 1:8, from about 1:1.1 to about 1:6, from about 1:1.1 to about 1:5, from about 1:1.1 to about 1:4, from about 1:1.1 to about 1:3, from about 1:1.1 to about 1:2, or from about 1:1.1 to about 1:1.5. In other embodiments the weight ratio of total amounts of thiol-based color-removing agent(s):keto acid(s) may be about 0.1, about 0.2, about 0.3, about 0.4, about 0.5, about 0.6, about 0.7, about 0.8, about 0.9, about 1, about 1.1, about 1.2, about 1.3, about 1.4, or about 1.5, including all ranges and subranges using any of the foregoing as upper and lower limits. The aforementioned ranges may be particularly chosen as weight ratios of thiol-based color removing agent(s):ketoglutaric acid.

### Clay Compounds

Emulsion compositions according to the disclosure optionally comprise at least one clay compound, although in at least some embodiments the emulsion compositions are free or essentially free of clay compounds. In some embodiments, the emulsion compositions comprise more than one clay compound, for example, at least two clay compounds, etc. Without intending to be limiting, it is believed that addition of one or more clay compounds may aid in preventing re-oxidation of the dyes, thus making the color-removal long lasting.

By way of non-limiting example, the clay compound may be chosen from kaolinite (also referred to interchangeably as kaolin), bentonite, magnesium aluminum silicate, hectorite, smectite, vermiculite, illite, chorite, halloysite, palygorskite, sepiolite, sesquioxide, imogolite, allophane, or combinations of two or more thereof. For example, in one embodiment, kaolin is chosen. In another embodiment, bentonite is chosen. In various embodiments, the clay compound comprises, consists essentially of, or consists of smectite, hectorite, kaolin, bentonite, magnesium aluminum silicate, or a combination of two or more thereof.

If present, the total amount of clay can range from about 0.1% to about 15%, such as from about 0.5% to about 12%, from about 1% to about 10%, or from about 2% to about 8% by weight, relative to the total weight of the composition. For example, the total amount of clay may range from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 3% to about 4%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 12%, from about 5% to about 11%, from about 5% to about 10%, from about 5% to about 9%, from about 5% to about 8%, from about 5% to about 7%, from about 5% to about 6%, from about 6% to about 12%, from about 6% to about 11%, from about 6% to about 10%, from about 6% to about 9%, from about 6% to about 8%, from about 6% to about 7%, from about 7% to about 12%, from about 7% to about 11%, from about 7% to about 10%, from about 7% to about 9%, from about 7% to about 8%, from about 8% to about 12%, from about 8% to about 11%, from about 8% to about 10%, from about 8% to about 9%, from about 9% to about 12%, from about 9% to about 11%, from about 9% to about 10%, from about 10% to about 12%, or from about 10% to about 11% by weight, relative to the total weight of the composition. For example, the total amount of clays may be about 1%, about 2%, about 3%, about 4%, about 5%, about 6%, about 7%, about 8%, about 9%, about 10%, about 11%, or about 12% by weight, relative to the total weight of the composition, including any range using any of the foregoing as upper and lower limits.

As a non-limiting example, the clay compound may comprise, consist essentially of, or consist of bentonite, kaolin, smectite, hectorite, or a combination of two or more thereof, and the total amount of clays may be about 1%, about 2%, about 3%, about 4%, about 5%, about 6%, about 7%, about 8%, about 9%, about 10%, about 11%, or about 12% by weight, relative to the total weight of the composition, or may be present in a range using any of the foregoing as upper or lower limits.

### Thickening Agents

Emulsion compositions according to the disclosure optionally comprise at least one thickening agent. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may range from about 0.001% to about 5%, such as from about 0.01% to about 4%, from about 0.1% to about 3.5%, from about 0.2% to about 3%, from about 0.3% to about 2.5%, from about 0.4% to about 2%, from about 0.5% to about 1.5%, or from about 0.5% to about 1% by weight, relative to the total weight of the composition.

### Amino Acids and Amino Sulfonic Acids

The emulsion compositions may optionally comprise at least one amino acid and/or amino sulfonic acid, although in at least some embodiments the compositions are free or essentially free of amino acids and/or amino sulfonic acids. Salts of amino acids and amino sulfonic acids may also be chosen, and are expressly included whether or not so stated.

Amino acids that may be chosen include, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, and amino hexyl benzene sulfonic acid. In some embodiments, the hair treatment compositions comprise at least one amino acid and at least one amino sulfonic acid. In some embodiments, the emulsion composition comprises arginine, serine, betaine, and/or taurine, for example arginine, betaine, or a combination thereof. For example, in some embodiments, arginine, serine, betaine, and/or taurine are the only amino acids and amino sulfonic acids present in the emulsion compositions, and in some embodiments, arginine and/or betaine are the only amino acids and amino sulfonic acids present in the emulsion compositions.

If present, the total amount of amino acids and/or amino sulfonic acids may range from about 0.01% to about 10%, such as from about 0.1% to about 8%, or from about 0.5% to about 6% by weight, relative to the total weight of the composition. For example, in some embodiments, the total amount of amino acid(s) and/or amino sulfonic acid(s) may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the composition. In some embodiments, the total amount of amino acid(s) and/or amino sulfonic acid(s) may be about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including all ranges and subranges using any of the foregoing as upper and lower limits.

### Organic Amine Compounds

In some embodiments, the emulsion compositions optionally comprise at least one organic amine compound, although in at least some embodiments the compositions are free or essentially free of organic amine compounds. Optionally, the emulsion compositions may comprise more than one organic amine compound, such as at least two organic amine compounds.

In various embodiments, alkanolamines, such as mono-, di-, or trialkanolamines, comprising from one to three identical or different C1-C4 hydroxyalkyl radicals, may be used. By way of example, organic amines such as monoethanolamine, diethanolamine, triethanolamine, monoisopropanolamine, diisopropanolamine, triisopropanolamine, N,N-dimethylethanolamine, 2-amino-2-methyl-1-propanol (AMP), 2-amino-2-methyl-1,3-propanediol, 3-amino-1,2-propanediol, 3-dimethylamino-1,2-propanediol, tris(hydroxymethyl)amino methane, guanidine, or combinations of two or more thereof may be chosen.

If present, the total amount of organic amine compounds may range from about 0.01% up to about 15%, such as up to about 14%, up to about 13%, up to about 12%, up to about 11%, up to about 10%, up to about 9%, up to about 8%, up to about 7%, up to about 6%, up to about 5%, up to about 4.5%, up to about 4%, up to about 3.5%, up to about 3%, up to about 2.5%, or up to about 2% by weight, relative to the total weight of the composition. For example, the total amount of organic amine compounds may range from about 0.01% to about 12%, from about 0.1% to about 10%, from about 0.5% to about 8%, or from about 1% to about 6% by weight, relative to the total weight of the composition. In at least some embodiments, the emulsion compositions comprise at least one organic amine compound, and have a total amount of organic amine compounds ranging from about 0.5% to about 12%, from about 0.5% to about 10%, from about 0.5% to about 8%, from about 0.5% to about 5%, from about 0.5% to about 4%, from about 0.5% to about 3%, from about 0.75% to about 12%, from about 0.75% to about 10%, from about 0.75% to about 8%, from about 0.75% to about 5%, from about 0.75% to about 4%, from about 0.75% to about 3%, from about 1% to about 12%, from about 1% to about 10%, from about 1% to about 8%, from about 1% to about 5%, from about 1% to about 4%, from about 1% to about 3%, from about 1.25% to about 12%, from about 1.25% to about 10%, from about 1.25% to about 8%, from about 1.25% to about 5%, from about 1.25% to about 4%, from about 1.25% to about 3%, from about 1.5% to about 12%, from about 1.5% to about 10%, from about 1.5% to about 8%, from about 1.5% to about 5%, from about 1.5% to about 4%, or from about 1.5% to about 3%, by weight relative to the total weight of the composition. In various embodiments, the total amount of organic amine compounds may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, or about 12% by weight, relative to the total weight of the composition, or may be present in any range using any of the foregoing as upper and lower limits.

### Auxiliary Components

Emulsion compositions according to the disclosure may optionally include one or more auxiliary components. Non-limiting examples include preservatives, fragrances such as parfum, pH adjusters, salts, antioxidants, vitamins (e.g. ascorbic acid, tocopherol), vitamin derivatives, botanical extracts, buffers, sequestering agents, and the like. In some embodiments, the emulsion compositions are free or essentially free of any of the aforementioned, e.g. the compositions may be free or essentially free of antioxidants, free or essentially free of ascorbic acid, etc. In particular embodiments, the compositions are free or essentially free of any component that would interfere with the composition being in emulsion form, e.g. such component is absent or, if present, is present in an amount below the amount that would interfere with the composition being in emulsion form.

The total amount of auxiliary components, if present, typically ranges from about 0.01% to about 15% by weight, based on the total weight of the composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

In some embodiments, the emulsion compositions may be free or essentially free of one or more of the following: ascorbic acid and salts thereof, dyes or pigments for coloring the hair, persulfates, peroxides, and/or ammonia. In yet other embodiments, the emulsion compositions may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of a total amount of ascorbic acid and salts thereof, persulfates, peroxides, or ammonia, or of a total amount of any combination of two or more thereof.

The compositions may be in any suitable emulsion form. For example, the compositions may be a liquid, a gel, a gel cream, a cream, etc. Advantageously, the emulsion compositions can be used as a one-part hair color removing composition, as compared to known systems for removing color from hair that require the application of multiple compositions to effectively remove color from the hair.

The pH of the emulsion composition is typically acidic, i.e. below 7. For example, compositions may have a pH ranging from about 1 to about 7, such as from about 2.5 to about 6.5. In some embodiments, the compositions have a pH ranging from about 2.5 to about 5, from about 2.75 to about 4.75, from about 3 to about 4.5, or from about 3 to about 4. For example, the pH of the emulsion composition may be about 3, about 3.25, about 3.5, about 3.75, about 4, about 4.25, or about 4.5.

### II. Methods

In various embodiments, the disclosure relates to methods for altering the color, tone, and/or shade of hair with the emulsion compositions described herein. The emulsions and methods can be used to remove some or all of certain dyes, while having minimal or no impact on other dyes. As such, the compositions can be targeted to remove certain dyes from the hair to achieve the consumer's desired result. For example, the methods may comprise methods of increasing the warmth of a hair color, for example by targeting dyes for removal that cause a color to be cooler. As a further example, the methods may comprise methods of increasing cool tones by targeting dyes for removal that cause a color to be warmer.

It is understood by those skilled in the art that the color, tone, and shade of hair are evaluated using the CIE L* a* b* system. In this system, the change in color, tone, and/or shade is determined by evaluating the color of hair after treatment (L*₂, a*₂, b*₂) compared to the color of the hair before treatment (L*₁, a*₁, b*₁). The overall change in color (ΔE) is defined as:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

In this system, the three parameters represent, respectively, the color intensity (L*), the green/red color axis (a*) (for example, higher a* is more red) and the blue/yellow color axis (b*) (for example, higher b* is more yellow). The higher the value of L*, the lighter the color, the higher the value of a*, the redder the color, and the higher the value of b*, the yellower the color. Additionally, the higher the value for ΔE, the greater the difference in color of treated hair relative to the color of the hair prior to treatment. Thus, methods may comprise altering the color, tone, and/or shade of hair by varying L*₂, a*₂, and/or b*₂ values, and/or by increasing the ΔE value relative to the hair prior to treatment.

The emulsion compositions and methods are thus able to achieve a desired hair color, tone, and/or shade even without, or with minimal, application of, subsequent hair coloring compositions. However, although the hair color, tone, and/or shade can be modified using compositions and methods according to the disclosure, in at least some embodiments the methods comprise an additional step of coloring or toning the hair after the hair is treated according to the disclosure.

The disclosure also relates to methods of removing artificial color from keratin fibers, especially hair that has been previously dyed with an oxidation dye. In particular, the emulsion compositions and methods according to the disclosure are particularly useful for removing darker colors from hair that may be otherwise difficult to remove with traditional hair-color removing compositions that do not use oxidizing agents. By way of non-limiting example, darker colors that can be removed in methods according to the disclosure include blacks, ash browns, browns, ash mochas, natural ash (blue or green), ash violets, violets, red violets, reds, red mochas, red browns, mahogany, red coppers, coppers, and golds.

The methods comprise applying an emulsion composition according to the disclosure onto the keratin fibers, e.g. hair that has been previously-dyed with at least one oxidative dye compound, optionally leaving the composition on the keratin fibers for a period of time (“processing time” or “leave-in period”), and subsequently rinsing or otherwise removing the composition from the keratin fibers. Thus, the methods may comprise a two-step method, which is simpler and more effective than known three- or more step methods where multiple compositions are applied to the hair for color removal before the hair is rinsed.

The appropriate leave-in period will be determined based on the color to be removed, and may in various embodiments last up to about 5 minutes, up to about 10 minutes, up to about 20 minutes, up to about 30 minutes, up to about 45 minutes, up to about 1 hour, up to about 2 hours, etc., such as from about 1 minute to about 60 minutes, from about 2 minutes to about 50 minutes, from about 5 minutes to about 45 minutes, or from about 10 minutes to about 40 minutes. It may be advantageous, in various embodiments, to leave the color-removing composition on the hair for at least about 15 minutes, and up to about 60 minutes. For example, the leave-in period may range from about 15 minutes to about 60 minutes, from about 20 minutes to about 60 minutes, or from about 25 minutes to about 60 minutes. Optionally, the hair may be covered, such as with a processing cap or aluminum foil, for at least a portion of the leave-in period.

Optionally, the hair to which an emulsion composition according to the disclosure has been applied may be heated during the leave-in period. For example, the hair may be heated using a hair dryer, hood dryer, or any other method for at least portion of the leave-in period. The hair may be heated using temperatures above room temperature, for example ranging from about 27° C. to about 90° C., from about 30° C. to about 80° C., from about 35° C. to about 70° C., or from about 40° C. to about 50° C. For example, an exemplary heating temperature range may be about 40° C. to about 45° C., such as about 40° C. to about 43° C.

In various embodiments, it may be advantageous to heat the treated hair for at least a portion of the leave-in period, with no heating for at least a portion of the leave-in period. By way of example, after the color-removing emulsion composition is applied to the hair, the treated hair may be left to process for a period of time at room temperature, followed by a period of time with application of heat. As an alternate example, after the color-removing composition is applied to the hair, the treated hair may be heated, followed by a period of time when the hair processes at room temperature. Further variations on such methods of alternating heating the treated hair and allowing the treated hair to process at room temperature may be used, such as, for example, heating followed by room temperature followed by heating, or processing at room temperature followed by heating followed by processing at room temperature, and so on.

In some embodiments, it may be advantageous to perform the methods described herein more than one time, in order to obtain the desired level of color removal. Because the emulsion compositions minimize damage to the hair and have decreased odor compared to known color-removal compositions, consumers are able to achieve the desired color removal without the disadvantages associated with such known compositions.

Surprisingly, the emulsion compositions and methods according to the disclosure effectively remove color from hair without the use of persulfates, peroxides, and/or ammonia, and without the damage that is typically associated with color removing compositions containing oxidizing agents.

It should be understood that compositions according to the disclosure are not compositions for coloring the hair. As such, the compositions are free or essentially free of dyes or pigments for coloring the hair, in particular of oxidative dye compounds. The compositions may optionally comprise a dye or pigment for coloring the composition, but it will be understood that the amount and/or type of dye or pigment will be chosen so that it does not impart color to the hair. Likewise, the methods according to the disclosure are not methods for imparting color to the hair.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

Unless otherwise defined for any specific embodiment, the term “substantially free” or “essentially free” as used herein means that there is less than about 5% by weight of a specific material added to a composition, based on the total weight of the composition. For example, the compositions may include less than about 4%, less than about 3%, less than about 2%, less than about 1.5%, less than about 1%, less than about 0.5%, less than about 0.1%, less than about 0.01%, less than about 0.001%, or less than about 0.0001% of the specified material. As such, it is contemplated that any component described herein for use in the compositions can be present in the compositions in amounts less than about 5%, less than about 4%, less than about 3%, less than about 2%, less than about 1%, less than about 0.5%, less than about 0.1%, less than about 0.01%, less than about 0.001%, or less than about 0.0001%, and the composition will be considered “substantially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

As used herein, “altering the color of hair,” “modifying the color of hair,” and variations thereof means that the color, tone, and/or shade of hair is changed by removing previously-applied hair dyes from the hair. Further, the terms “color removal,” “hair color-removing,” and variations thereof should be understood to refer to removal of some or all of previously-applied hair dyes from the hair. It should be understood that removal of artificial color can include removal of all color, or can include removal of various tones of color. Therefore, in some embodiments, overall change in the hair color will be observed (e.g. greater ΔE), while in other embodiments even if the overall color change is not considered significant, variation in one or more of the L*, a*, or b* values will be observed.

As used herein, “artificial color” means color that has been imparted to the hair by means of prior application of a composition capable of changing the color of hair, for example with permanent, semi-permanent, or demi-permanent hair dye compositions. In various embodiments the artificial color that is removed from the hair is color that has been imparted to the hair by one or more oxidative dye compounds.

As used herein, the terms “odor-free,” “minimal odor,” “no odor,” “reduced odor,” or the like are intended to mean the odor that is typically associated with color removing compositions containing thiolactic acid color removers, which odor hair color professionals and consumers typically find pungent and/or offensive.

As used herein, “thiol-based” is used according to its ordinary meaning to signify that the compound has an R—SH (thiol) group, where R is an alkyl group or other organic substituent.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention.

### EXAMPLES

The following Examples are intended to be non-limiting and explanatory in nature only. In the Examples, amounts are expressed in percentage by weight (wt %) of active materials, relative to the total weight of the composition, unless otherwise indicated.

In these Examples, the change in the color of hair is evaluated with the CIE L* a* b* system using Colorshot MS, where the change is determined by evaluating the color of the hair after treatment (L*₂, a*₂, b*₂) compared to the color of the hair before treatment (L*₁, a*₁, b*₁). The change in color (ΔE) is calculated as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} .$$

### Example 1—Compositions

Emulsion compositions 1A-1C according to the disclosure were prepared as shown in Table 1A, and comparative compositions C1-C3 were prepared as shown in Table 1B. Compositions C1-C3 were in the form of aqueous simplex solutions.

TABLE 1A
Emulsion Compositions

| | 1A | 1B | 1C |
|---|---|---|---|
| THICKENERS (xanthan | 0.7 | 0.7 | |
| and/or sclerotium gums) | | | |
| AMINOMETHYL | 3.6 | 3.0 | |
| PROPANOL | | | |
| ETHANOLAMINE | | | 3.0 |
| DICETYL PHOSPHATE | | | 0.7 |
| KETOGLUTARIC ACID | 10.0 | 5.0 | 10.0 |
| CETETH-10 PHOSPHATE | | | 0.6 |
| 2-OLEAMIDO-1,3- | | | 0.01 |
| OCTADECANEDIOL | | | |
| SILICONE OILS | 2.0 | 2.0 | |
| ISOPROPYL ALCOHOL | 0.7 | 0.7 | |
| CETEARYL ALCOHOL | 5.0 | 5.0 | 10.8 |
| BEHENTRIMONIUM | 3.2 | 3.2 | |
| CHLORIDE | | | |
| THIOLACTIC ACID | | | 5.0 |
| THIOSALYCYLIC ACID | 5.0 | 2.5 | |
| MINERAL OIL | | | 3.0 |
| STEARETH-20 | 1.0 | 1.0 | |
| PPG-5-CETETH-20 | | | 3.0 |
| FRAGRANCE | 0.8 | 0.8 | 1.0 |
| ADDITIVES (pH | ≤2 | ≤2 | ≤2 |
| adjuster/buffer, preservative, | | | |
| chelator, emollient) | | | |
| SOLVENTS (water and non- | QS to 100 | QS to 100 | QS to 100 |
| aqueous solvents) | | | |

TABLE 1B
Comparative Compositions

| | C1 | C2 | C3 |
|---|---|---|---|
| FRAGRANCE | 1.0 | | 1.0 |
| XANTHAN GUM | 0.7 | 0.7 | 0.7 |
| BENTONITE | | | 5.0 |
| ETHANOLAMINE | 2.1 | 2.1 | 1.7 |
| KETOGLUTARIC ACID | 10.0 | 5.0 | 10.0 |
| ARGININE | 3.0 | | 3.0 |
| THIOLACTIC ACID | | | 5.0 |
| THIOSALYCYLIC ACID | 5.0 | 2.5 | |
| SODIUM LAURYL SULFATE | 1.5 | 1.5 | 1.5 |
| SOLVENTS (water and non- | QS to 100 | QS to 100 | QS to 100 |
| aqueous solvents) | | | |

### Example 2—Evaluation of Hair Color Removal and Hair Color Alteration

The following studies were performed to evaluate the efficacy of emulsions according to the disclosure to remove color and to alter the color, tone, and/or shade of hair that has previously been dyed with oxidative hair dye compositions.

Three sets of seven swatches of permed, ˜90% grey hair were colored with one of three different commercially-available oxidative hair dye compositions, each having different combinations of oxidative dye compounds (21 swatches total, 7 per color). The oxidative hair dye composition was mixed in a 1:1 ratio with 20V hydrogen peroxide developer to form a hair dye mixture, and the hair dye mixture was applied to the swatches at a rate of about 3 grams per gram of hair. The swatches were left to process at room temperature for about 35 minutes, rinsed, shampooed with a commercial shampoo, rinsed again, and dried.

Once the swatches were dry, six of seven swatches in each set were treated as follows. One of emulsion compositions 1A-1C or comparative compositions C1-C3 was applied to one swatch from each set at a rate of about 2 grams per gram of hair. After the swatches were allowed to process for 30 minutes at about 33° C., the swatches were rinsed for about 30 seconds, shampooed with a commercial shampoo, rinsed, shampooed again, rinsed again, and then dried. One swatch per set had no color-removing composition applied, and was the color control.

The color of each of the swatches was then evaluated with the CIE L* a* b* system, to evaluate the effectiveness of color removal. Table 2A shows the ΔE values for the treated swatches, relative to the color control, and Table 2B shows the L*, a*, and b* results.

TABLE 2A
Measured ΔE Results

| | Set 1 | Set 2 | Set 3 |
|---|---|---|---|
| Control | — | — | — |
| 1A | 45.19 | 10.67 | 38.62 |
| C1 | 41.85 | 13.71 | 42.57 |
| 1B | 44.31 | 6.72 | 31.13 |
| C2 | 41.85 | 6.61 | 20.86 |
| 1C | 46.05 | 33.88 | 43.82 |
| C3 | 49.23 | 24.44 | 40.76 |

TABLE 2B
Measured L*, a*, b* Results

| | Set 1 | | | Set 2 | | | Set 3 | | |
|---|---|---|---|---|---|---|---|---|---|
| | L* | a* | b* | L* | a* | b* | L* | a* | b* |
| 1A | 38.26 | 10.76 | 30.39 | 11.2 | 17.23 | 8.72 | 47.37 | 12.81 | 23.85 |
| C1 | 31.57 | 19.11 | 28.26 | 12.06 | 19.81 | 10.11 | 52.63 | 13.25 | 22.34 |
| 1B | 39.12 | 14.22 | 26.68 | 9.73 | 14.22 | 6.35 | 44.18 | 22.15 | 22.08 |
| C2 | 34.14 | 16.76 | 27.13 | 9.46 | 14.21 | 6.14 | 44.11 | 23.04 | 22.68 |
| 1C | 43.15 | 11.75 | 25.32 | 23.65 | 28.19 | 26.67 | 53.87 | 12.4 | 20.04 |
| C3 | 45.76 | 7.39 | 28.51 | 19.10 | 23.94 | 19.29 | 49.41 | 11.57 | 22.58 |

The results in Table 2A show that oxidative hair dye can be more effectively removed with emulsion compositions according to the disclosure. The results show an increase in ΔE of hair treated with compositions 1A or 1B compared to hair treated with composition C1 or C2 in Set 1, show an increase in ΔE of hair treated with composition 1C compared to hair treated with composition C3 in Set 2, and show an increase in ΔE of hair treated with compositions 1B or 1C compared to hair treated with composition C2 or C3 in Set 3. These results are significant because composition pairs 1A and C1, 1B and C2, and 1C and C3, have the same amounts of color-removing agents, respectively. The difference in each pair is that compositions 1A, 1B, and 1C were in the form of emulsions, but compositions C1, C2, and C3 were not.

Moreover, the results in Table 2B demonstrate that compositions according to the disclosure can be used to alter the color, tone, and/or shade of hair in a controlled manner. For example, as the data in Set 3 show, although composition 1C had a lower overall change in color than composition C3 (lower ΔE), relative to composition C3 composition 1C provided a more red (higher a* value) and less yellow (lower b* value) tone. Similarly, as the results for Set 2 show, although composition 1C had a greater overall change in color than composition C3 (higher ΔE), relative to composition C3 composition 1C provided a lighter shade (higher L* value) with a more red (higher a* value) and less yellow (lower b* value) tone.

These data demonstrate that the compositions and processes according to the disclosure can be used to modify hair color in a targeted manner, unlike traditional hair color-removing compositions. Compositions according to the disclosure have the unique ability to fade-on-tone and deliver the desired color, tone, and/or shade, even in the absence of subsequent hair color applications.

### Example 3—Evaluation of Odor Control

Consistent with how such evaluations are typically done, performance of odor control was evaluated olfactorily by a panel of fragrance experts (n=5) who evaluated the odor of compositions 1C and C3, each with 5% thiolactic acid as a color-removing agent, and ranked the odor according to the criteria in Table 3A.

TABLE 3A
Rating Criteria

| Rating | Odor Criteria |
|---|---|
| 1 | Very unpleasant |
| 2 | Unpleasant, intense |
| 3 | Tolerable, some malodor |
| 4 | Good odor, mild malodor |
| 5 | Pleasant, no malodor |

The results of the odor evaluation are shown in Table 3B.

TABLE 3B
Results

Rating

| Panelist | 1C | C3 |
|---|---|---|
| 1 | 4 | 3 |
| 2 | 4 | 2.5 |
| 3 | 4 | 3.75 |
| 4 | 4.5 | 2.5 |
| 5 | 4 | 3 |
| Average | 4.1 | 2.95 |

The data in Table 3 show that the use of thiolactic acid in an emulsion composition unexpectedly reduces the malodor that is commonly associated with thiolactic acid-containing color-removing compositions.

The results of Examples 2 and 3 together demonstrate that color-removing compositions according to the disclosure effectively remove artificial color from hair, while minimizing the offensive odor typical of traditional hair color removing compositions and processes. These results are surprising because it was not previously known that providing the compositions in emulsion form would increase the efficacy of hair color-removing agents, or that it would provide additional advantageous benefits such as reduced odor.

### Example 4—Additional Emulsion Compositions

Emulsion compositions 2A-2D can also be prepared as shown in Table 4, and are likewise expected to provide similar hair color removing, hair color altering, and reduced odor benefits.

TABLE 4

| | 2A | 2B | 2C | 2D |
|---|---|---|---|---|
| THIOSALICYLIC ACID | | 5.0 | 2.0 | |
| THIOLACTIC ACID | 4.0 | | | 8.5 |
| ARGININE | | 2.5 | 1.0 | 1.5 |
| BETAINE | 1.9 | 1.6 | | |
| PROLINE | | | | 0.5 |
| XANTHAN GUM | 0.7 | 0.7 | | |
| HYDROXYPROPYL | | | 1.5 | 1.0 |
| CELLULOSE | | | | |
| BENTONITE | | 5.0 | | |
| KAOLIN | | | 3.0 | |
| ETHANOLAMINE | | 3.7 | | 1.0 |
| AMINOMETHYL PROPANOL | 4.5 | | | |
| SODIUM LAURYL SULFATE | 5.0 | 5.0 | | |
| SODIUM LAUROYL METHYL | | | 2.5 | 7.5 |
| ISETHIONATE | | | | |
| CITRIC ACID | | | 5.0 | |
| α-KETOGLUTARIC ACID | | 10.0 | | 2.0 |
| FATTY COMPOUNDS | 9.5 | 7.0 | 1.5 | 5.0 |
| (silicone oils and/or plant oils) | | | | |
| ADDITIVES (fragrances, pH | ≤5 | ≤5 | ≤5 | ≤5 |
| adjuster, vitamins, plant | | | | |
| extracts, preservatives, etc.) | | | | |
| SOLVENTS (water and non- | QS | QS | QS | QS |
| aqueous solvents) | to 100 | to 100 | to 100 | to 100 |

The above Examples demonstrate that compositions and methods according to the disclosure effectively remove various oxidative hair dyes from hair, allowing the hair color, tone, and/or shade to be altered in a controlled manner, while surprisingly minimizing or reducing associated unpleasant odor during the process.

These compositions and processes are unique in that they can provide a targeted method for removing hair color to provide consumers with choices in resulting color, even without the need to backfill the color to arrive at the desired result. The compositions and processes can be particularly useful when similar tonality, rather than an entirely different hair color, is desired, as the data in the above Examples demonstrate.

Moreover, the Examples show that the compositions are processes are particularly useful for removing darker colors, which tend to be more difficult to remove with color-removing agents that are not oxidizing agents.

1. A hair-color altering emulsion composition comprising:
a) at least one hair-color removing agent;
b) at least one surfactant;
c) at least one fatty compound;
d) water; and
e) optionally at least one organic acid and/or salt thereof,
wherein the total amounts of surfactants, fatty compounds, and water are sufficient to maintain the composition in the form of an emulsion.

2. The hair-color altering emulsion composition according to claim 1, comprising at least one hair-color removing agent chosen from thiosalicylic acid, thiolactic acid, and/or salts thereof.

3. The hair-color altering emulsion composition according to claim 1, wherein the total amount of hair-color removing agents ranges from about 0.5% to about 20% by weight, relative to the total weight of the composition.

4. The hair-color altering emulsion composition according to claim 1, comprising at least one keto acid and/or salt thereof, wherein the total amount of keto acids and salts thereof ranges from about 0.1% to about 20% by weight, relative to the total weight of the composition.

5. The hair-color altering emulsion composition according to claim 1, wherein the total amount of surfactants ranges from about 0.5% to about 12% by weight, based on the total weight of the composition.

6. The hair-color altering emulsion composition according to claim 1, wherein the total amount of fatty compounds ranges from about 0.1% to about 15% by weight, based on the total weight of the composition.

7. The hair-color altering emulsion composition according to claim 1, further comprising at least one additional component chosen from clays, amino acids and/or salts thereof, amino-sulfonic acids and/or salts thereof, organic amine compounds, or combinations of two or more thereof.

8. The hair-color altering emulsion composition according to claim 6, comprising at least one organic amine compound.

9. The hair-color altering emulsion composition according to claim 1, wherein the composition has a pH ranging from about 1 to about 6.

10. A method for altering the color of hair, the method comprising applying to the hair an emulsion composition comprising:
a) at least one hair-color removing agent;
b) at least one surfactant;
c) at least one fatty compound;
d) water; and
e) optionally at least one organic acid and/or salt thereof,
wherein the total amounts of surfactants, fatty compounds, and water are sufficient to maintain the composition in the form of an emulsion.

11. The method according to claim 10, wherein the emulsion composition comprises at least one hair-color removing agent chosen from thiosalicylic acid, thiolactic acid, and/or salts thereof.

12. The method according to claim 10, wherein the total amount of hair-color removing agents in the emulsion composition ranges from about 0.5% to about 20% by weight, relative to the total weight of the composition.

13. The method according to claim 10, wherein the emulsion composition comprises at least one keto acid and/or salt thereof, wherein the total amount of keto acids and salts thereof ranges from about 0.1% to about 20% by weight, relative to the total weight of the composition.

14. The method according to claim 10, wherein the total amount of surfactants in the emulsion composition ranges from about 0.5% to about 12% by weight, based on the total weight of the composition.

15. The method according to claim 10, wherein the total amount of fatty compounds in the emulsion composition ranges from about 0.1% to about 15% by weight, based on the total weight of the composition.

16. The method according to claim 10, wherein the emulsion composition further comprises at least one additional component chosen from, clays, amino acids and/or salts thereof, amino-sulfonic acids and/or salts thereof, organic amine compounds, or combinations of two or more thereof.

17. The method according to claim 16, wherein the emulsion composition comprises at least one organic amine compound.

18. The method according to claim 10, wherein the emulsion composition is left on the hair for a period of time ranging from about 5 minutes to about 45 minutes and then rinsed from the hair.

19. The method according to claim 10, wherein the hair to which the emulsion composition is applied comprises at least one oxidative dye compound.

20. A hair-color altering emulsion composition comprising:
a) at least one color-removing agent chosen from thiosalicylic acid, thiolactic acid, and/or salts thereof;
b) at least one surfactant;
c) at least one fatty compounds
d) water; and
e) at least one additional compound chosen from keto acids and/or salts thereof, thickening agents, carboxylic acids and/or salts thereof, amino acids and/or salts thereof, amino-sulfonic acids and/or salts thereof, organic amine compounds, or combinations of two or more thereof,
wherein the total amounts of surfactants, fatty compounds, and water is sufficient to maintain the composition in the form of an emulsion.
